(19) United States
(12) Patent Application Publication
(10) Pub. No.: US 2024/0315592 A1
(43) Pub. Date: Sep. 26, 2024

(54) SYSTEM FOR DETECTION OF BIOMARKERS IN AIR EXHALED FROM PATIENT'S LUNGS, METHOD FOR DETECTION OF BIOMARKERS IN AIR EXHALED FROM PATIENT'S LUNGS AND SYSTEM FOR DETECTION OF GASES, ESPECIALLY BIOMARKER GASES

(71) Applicant: UNIWERSYTET WARSZAWSKI, Warszawa (PL)

(72) Inventors: Tadeusz STACEWICZ, Warszawa (PL); Mateusz WINKOWSKI, Warszawa (PL)

(73) Assignee: UNIWERSYTET WARSZAWSKI, Warszawa (PL)

(21) Appl. No.: 18/578,995

(22) PCT Filed: Jul. 11, 2022
(86) PCT No.: PCT/PL2022/050045

§ 371 Date: Jan. 12, 2024

(30) Foreign Application Priority Data
Jul. 12, 2021 (PL) P.438444

(51) Int. Cl. A61B 5/08; A61B 5/097; A61B 10/00; G01N 21/3504

(52) U.S. Cl. CPC A61B 5/082; A61B 5/097; G01N 21/3504; A61B 2010/0087; G01N 2201/06113; G01N 2201/0636

(57) ABSTRACT

The invention relates to a system for the detection of biomarkers in the air exhaled from patient's lungs, comprising a leak-proof multipass cell provided with a gas inlet and outlet, into which a sample of the exhaled air is introduced by means of a pneumatic system, wherein the multipass cell is provided with one laser whose light beam is directed along a coaxial optical path to the multipass cell comprising at least two concave mirrors arranged in parallel and a gas inlet and outlet, which is filled with a sample of the analyzed exhaled gas, wherein the pneumatic system is connected to the gas inlet and outlet of the multipass cell by means of lines supplying the exhaled air, characterized in that the pneumatic system comprises a preliminary container of the exhaled air comprising an air inlet and outlet, wherein the air outlet additionally comprises an air flow meter, the preliminary container is further connected by an elastic line supplying the air to the multipass cell on one side and on the other side to a pump, baratron, and also the air outlet of the multipass cell. The invention also relates to a method for the detection of biomarkers in the air exhaled from patient's lungs, with the use of such a system and to a system for the detection of gases, especially biomarker gases, using multiplexing and demultiplexing of optical signals.

### TECHNICAL FIELD

The invention relates to a system for the detection of biomarkers in the air exhaled from patient's lungs and a method for the detection of biomarkers in the air exhaled from patient's lungs with the use of laser spectroscopy. The invention also relates to a system for the detection of gases, especially biomarker gases, using the multiplexing and demultiplexing of optical signals. More specifically, the invention relates to a method for combining and then splitting light signals used in optics, optoelectronics, fiber-optic and telecommunication technologies, and laser spectroscopy.

### BACKGROUND ART

Laser spectroscopy (laser absorption spectrometry, LAS) is a branch of spectroscopy using laser radiation to study the properties of matter. Laser spectroscopy enables, among others, a very sensitive (it detects trace amounts of impurities, at ppm level) and quick (in several, ten or so seconds) detection of chemical compounds in the gaseous phase [U.S. Pat. No. 7,612,885B2]. Laser spectroscopy is also used for the studies of interactions between light and matter, to study interactions between atoms as well as intramolecular and intermolecular interactions [W. Demtroder, Spektroskopia laserowa [Laser spectroscopy], PWN, Warszawa, 1993].

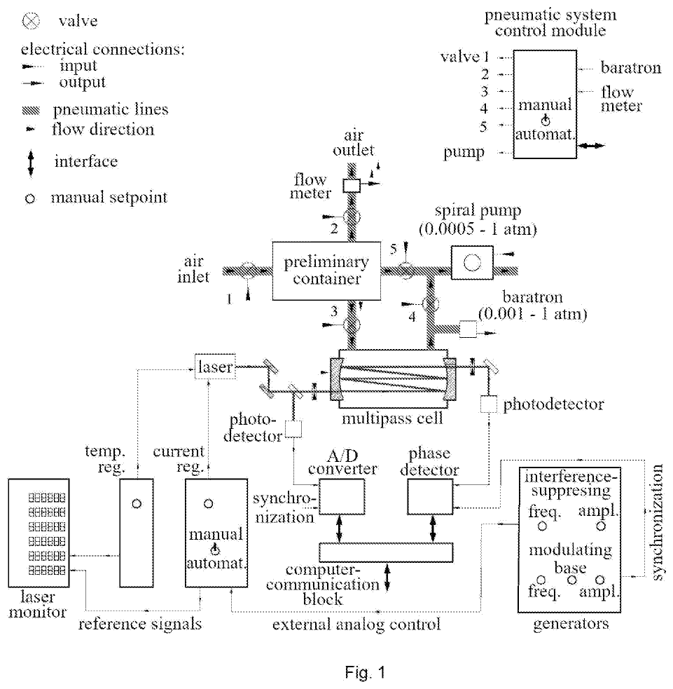

Laser spectroscopy systems often require the use of light beams produced simultaneously by several lasers (each operating at a different wavelength—US20050122523A1) and it is necessary that the beams travel coaxially i.e. they are not split spatially. In typical applications, the light stream prepared in this way gets into a spectroscopic system wherein certain information is encoded in individual beams. Retrieval of this information in channels corresponding to predetermined wavelengths requires demultiplexing, i.e. separation. One example is provided by systems for gas diagnostics. [(a) D. B. Oh, M. E. Paige, D. S. Bomse, Applied Optics, 1998, 37, 2499-2501; (b) C. Dong et al., Chin. Phys. Lett., 2006, 23, 2446-2449; (c) T. Cai, G. Wang, W. Zhang, X. Gao, Measurement, 2012, 45, 2089-2095], in which the concentration of impurities is determined by measuring light absorption (FIG. 1). Coaxiality of several beams is particularly important when using systems of ultrasensitive spectroscopy, such as multipass spectroscopy (see below) or Cavity Ring Down Spectroscopy (CRDS) in applications for simultaneous detection of several components [U.S. Pat. No. 7,541,586B2].

Combination of several laser beams (multiplexing) into one coaxial stream of light can be carried out by various methods, e.g. by means of beam splitters, i.e. dichroic mirrors—see FIG. 1, or polarization splitters. A radiation beam prepared in such a way, travelling through a spectroscopic system interacts with a medium filling the absorption chamber (FIG. 1). In this way, certain information is encoded into individual wavelengths. In the absorption studies it is information about the concentration of individual components in a sample. Decoding of this information requires demultiplexing in order to determine the level of radiation weakening at each wavelength. Typically, this procedure is repeated by splitting laser beams, e.g. by means of a dichroic or polarization splitter, into independent photodetectors, each of which produces an output signal corresponding to the radiation intensity at the corresponding wavelength.

Nevertheless, with a greater number of laser beams, the optical system becomes complicated. Even with two lasers there are problems with the coaxiality and stability of the beams at a longer travelling distance (several dozen-several hundred meters). These are limitations resulting from the finite precision and stability of optical and optomechanical elements. Difficulties related therewith are particularly important in the case of multipass spectroscopy.

Another problem relates to demultiplexing (splitting) of information carried at individual wavelengths in the coaxial beam after it has passed through a spectroscopic system. Splitting of beams corresponding to different wavelengths is carried out by means of dichroic mirrors, or other optical elements, such as polarization splitters, diffraction gratings or prisms. Subsequently, the beams are directed to independent photodetectors operating at different detection channels (different wavelengths or different light polarizations). This approach is ineffective and is characterized by numerous disadvantages, because splitting by means of the above mentioned methods is imperfect and burdened with interferences between channels. Reduction of interferences requires additional use of narrowband filters (e.g. interference and/or color filters), which leads to complexity of an optical system, weakening of optical signals and worsening of the strength of signals-to-strength of interference signals ratio.

Determination of the absorption coefficient [α] of samples of matter is usually performed in one-pass spectroscopy systems. It consists in measuring the weakening of radiation which passes once through a measurement cell having a length [z] and containing an absorber. If the intensity of radiation reaching the cell (monitored by an input photodetector (IN)) is [I_{IN}], the intensity registered by the photodetector at the output [I_{OUT}] has the value:

$${I}_{O U T} = {I}_{IN} \cdot {e}^{( - α \cdot z )}$$

On the basis of the above equation the absorption coefficient [α] can be determined:

$$α = \frac{ln ( \frac{{I}_{IN}}{{I}_{OUT}} )}{z}$$

In practice, it is difficult to achieve high measurement sensitivity. It results from the analysis of the above equation that, when the values [I_{IN}] and [I_{OUT}] differ insignificantly, the measurement error strongly increases. Increasing measurement sensitivity consists in lengthening the optical path in the measurement cell by the use of multipass spectroscopy. It consists in the use of an absorption cell ended with mirrors. At the ends of the absorption cell, two mirrors are placed, between which the light beam introduced into the cell is reflected multiple times therein. In this way a light path having several dozen or even several hundred meters can be obtained in the cell and thus an increased sensitivity of the system. In both cases, it is possible to achieve a further improvement of measurement sensitivity by using amplitude modulation or laser wavelength modulation and phase detection [W. R. Watkins, Applied Optics, 1976, 15, 2-19].

Cavity Ring Down Spectroscopy (CRDS) is used to measure small absorption coefficients of gas mixtures. The measurement is performed by means of an optical resonator (cavity) containing a sample of examined gas. The resonator (usually a Fabry-Pérot interferometer) is constructed of two mirrors having a very high reflection coefficient [“Laser Beams and Resonators”, H. Kogelnik and T. Li, Applied Optics, 1966, 5 (10), 1550-1567]. A laser beam having a length adjusted to the absorption line of the examined gas, or its component being sought, is introduced into the resonator by one of the mirrors (see FIG. 3a). In order to measure the absorption coefficient, the Quality factor (or Q factor) of the cavity is determined, which is the lesser, the bigger the absorption coefficient of matter filling the resonator is. The absorption coefficient is determined as result of comparing the quality of the resonator with the case where it is filled with a reference gas which does not contain an absorber. [A. O'Keefe, D. A. G. Deacon, Review of Scientific Instruments, 1988, 59, 2544-2550]. Radiation coming out from the resonator is registered by a photodetector.

There are several methods for determining the quality factor of an optical resonator. Most frequently, for this purpose, laser radiation with amplitude modulated to a rectangle-shaped signal is introduced to the cavity. Due to multiple reflections between the mirrors (provided that the geometry and frequency of laser radiation are adjusted to the cavity mode), light is stored in the resonator at each impulse. As a result of the storage, radiation coming out from the resonator and registered by a photodetector and an oscilloscope has edges that change with time {t] in accordance with the exponential functions: ˜exp(−t/T) (trailing edge) and ˜[1−exp(−t/T)] (leading edge), wherein [T] stands for the time of light storage in the cavity.

In the case where there is an absorber in the cavity, the storage time [T_{A}] becomes shorter: [T_{A}<T]. By registering signals by means of a digital oscilloscope and determining the times [T] and [T_{A}] one can determine the resonator quality in each of these cases [Q=v·T] ([v] stands for radiation frequency) and determine the absorption coefficient of matter that fills the cavity:

$$α = \frac{1}{c} ( \frac{1}{{τ}_{A}} - \frac{1}{τ} ) \qquad ( 1 )$$

wherein [c] stands for light speed.

Another method consists in replacing a digital oscilloscope in the measurement system with a phase voltmeter that registers first harmonics and phases of input and output signal. Due to the storage of radiation in the cavity, a time shift [Δt] (and thus a phase shift [φ]) appears between the harmonics. The Fourier analysis of signals enables finding a formula determining the value of the shift: φ=2πfΔt=arctg(2πfτ), wherein [f] stands for modulation frequency. By putting the above dependencies together with formula (1), and having determined analogous phase shifts [φ] and [φ_{A}], it is possible to determine the resonator quality and absorption coefficient of matter that fills the cavity.

In the prior art there are known systems for monitoring biomarkers in the air exhaled by the patient; e.g. [US2020100702A1] describes a system and method for a discrete analysis of the exhaled air in a sample, in a cost-effective and non-invasive manner. The manual system for the analysis of breath is provided with a main housing, a mouthpiece, a lid, a sampling chamber, a sensor and a microprocessor.

WO2017216794A1 describes a system and method for diagnosing, screening or monitoring of a stage of development of a disease by analyzing a breath of a test subject using a sensor set in conjunction with automatic pattern recognition analyzer, wherein the pattern recognition analyzer receives output signals of the sensor set, compares them to disease-specific patterns derived from a database of response patterns of the sensor set to exhaled breath of subjects with known diseases, wherein each of the disease-specific patterns is characteristic of a particular disease, and selects a closest match between the output signals of the sensor set and the disease-specific pattern.

For the purposes of description of this invention, light beams should be identified with the physical optical path along which these optical beams travel. The optical path may be provided by optical fibers as well as the air. Moreover, for the purposes of description of this invention, where appropriate, the terms ‘optical path’ and ‘optical trajectory’ may be used interchangeably.

### Solution of Technical Problem

The invention solves the problem concerning detection of biomarkers in the air exhaled from patient's lungs in real time of patient examination.

### DISCLOSURE OF INVENTION

The invention provides a system for the detection of biomarkers in the air exhaled from patient's lungs, comprising a leak-proof multipass cell provided with a gas inlet and gas outlet, into which a sample of exhaled air is introduced by means of a pneumatic system, wherein the multipass cell is provided with one laser whose light beam is directed along a coaxial optical path to the multipass cell comprising at least two concave mirrors arranged in parallel, and a gas inlet and outlet, which is filled with a sample of the analyzed exhaled air, wherein the pneumatic system is connected to the gas inlet and outlet of the multipass cell by means of lines supplying the exhaled air, characterized in that the pneumatic system comprises a preliminary container of the exhaled air comprising an air inlet and air outlet, wherein the air outlet is additionally provided with an air flow meter, the preliminary container is further connected by an elastic line supplying the air to the multipass cell on one side and on the other side with a pump, baratron and air outlet from the multipass cell.

Preferably, the pump is a spiral pump, especially an oil-free pump, adjusted to reduce the pressure within the range from 1 atm to 0.0005 atm. An advantage of the oil-free pump is that its use eliminates the necessity to add oil and avoids contamination of the system with oil vapors.

Preferably, the baratron is adjusted to measure reduced pressure within the range from 1 atm to 0.001 atm.

Preferably, the preliminary container comprises elastic lines supplying the analyzed exhaled air, wherein each of the lines is independently provided with an independently controlled valve.

Preferably, the elastic line supplying the analyzed exhaled air additionally comprises a valve between the pump and the baratron.

Preferably, the laser is connected by a coaxial optical path to the multipass cell and to one photodetector in front of the multipass cell and another photodetector behind the multipass cell.

Preferably, the pneumatic module is controlled by means of a pneumatic system control module of valves and the pump.

Preferably, the laser is connected by electric lines to a laser temperature control module and laser operation control module, and both these modules are electrically connected to a laser status monitoring module.

Preferably, the laser is a laser emitting monochromatic light beams of different wavelengths.

Preferably, the laser is a laser emitting a monochromatic light beam having a wavelength adjusted to the edge of the absorption line of formaldehyde (HCOH) within the range of 3595.77-3596.20 nm, and a laser emitting a light beam adjusted to the edge of the absorption line of ethane (C₂H₆) within the range close to 3336.8 nm.

Preferably, 1046 Hz rectangular signals are used to modulate the laser emitting light having a wavelength within the range of 3595.77-3596.20 nm, whereas 1429 Hz rectangular signals are used to modulate the laser emitting light having a wavelength within the range close to 3336.8 nm.

Modulation consists in a periodic change of laser wavelength within the range of the absorption spectrum of a detected biomarker. The wavelength of a laser diode used in this system depends on the amperage of current supplied thereto. Therefore, it is periodically changed (modulated) due to the supply of current comprising-apart from a constant component—also a small variable component with appropriate amplitude.

Preferably the distance between the mirrors in the multipass cell is at least 1 cm, preferably 50 cm.

The system according to the invention enables detection of any biomarker in the air exhaled from patient's lungs in real time. Each biomarker requires the use of a specific laser generating radiation having a wavelength adjusted to the absorption lines of the molecules of the analyzed biomarker.

In accordance with the invention, experimental tests have been carried out with the use of the system for the detection of biomarkers according to the invention, which tests have shown, using the exemplary, representative, biomarkers present in the exhaled air, such as methane, ethane, formaldehyde, or water vapor, that it is possible to detect any biomarker in the air exhaled from patient's lungs with use of the system according to the invention. The experimental results have been obtained with the use of systems comprising lasers adjusted to the above mentioned biomarkers and sensors of formaldehyde (the obtained detection sensitivity was 6 ppb) and ethane (the obtained detection sensitivity was 1 ppb). The use of the system for the detection of biomarkers according to the invention also enables detection of other biomarkers, such as water vapor (0.1%) and methane (0.05 ppm). The obtained detection sensitivities were determined using calibrated mixtures of the indicated gases (biomarkers) with the air, prepared by renowned firms (Messer). An advantage of the system for the detection of biomarkers according to the invention is a high selectivity of measurements, understood as insensitivity to the influence of other biomarkers (biomarker gases).

The invention also provides a method for the detection of biomarkers in the air exhaled from patient's lungs with the use of the system for the detection of biomarkers according to the invention as defined above, comprising the following stages:
(i) preparing the system to take a sample of exhaled air and measure a biomarker;
(ii) taking a biomarker sample;
(iii) filling the multipass cell;
(iv) adjusting pressure in the multipass cell;
(v) measuring the content of biomarkers in the air sample;
(vi) emptying the pneumatic system of the examined air

Preferably, in stage (i):
a) the preliminary container is filled with the air under atmospheric pressure;
b) there is no flow of gas through the flow meter;
c) the multipass cell is being pumped out to the pressure limit of the pump (0.001 atm);
d) the pump is turned off;
e) all valves (1-5) are closed;
f) PSCM generates the signal “READY”.

Preferably, in stage (ii):
a) valves 1 and 2 are open;
b) the remaining valves (3÷5) are closed;
c) the pump is turned off;

Preferably, in stage (iii):
a) valves 1, 2 and 4 and 5 are closed;
b) valve 3 (with a flow limiter) is being opened and the gas from the preliminary container flows to the multistage cell (FIG. 5);
c) pressure build-up in the multipass cell is monitored by the baratron.

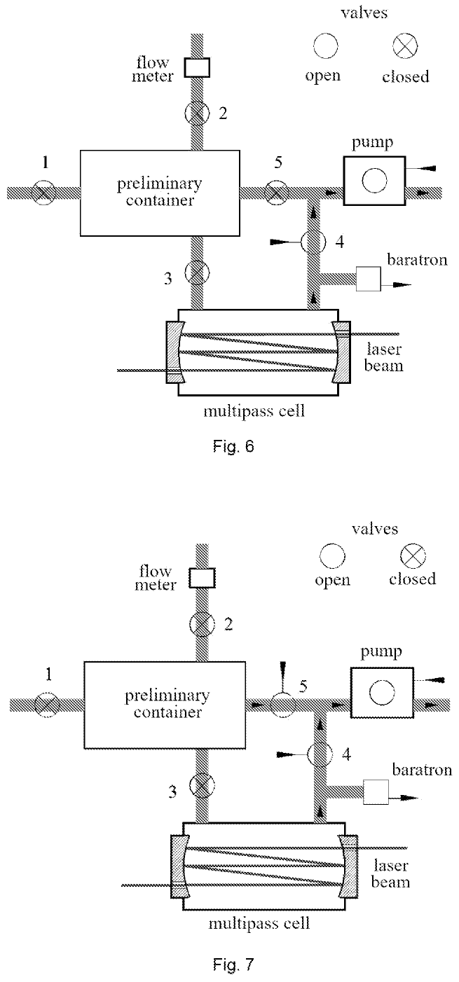

Preferably, in stage (iv):
a) valves 1÷3 and 5 are closed;
b) valve 4 (with the flow limiter) is being opened;
c) the pump is being turned on—the pressure in the multipass cell is being reduced (FIG. 6);
d) the pressure in the multipass cell is monitored by the baratron. In the case when the measurement value is achieved (for example, amounting to 0.1 atm for ethane and 0.01 atm for formaldehyde), valve 4 is being closed and the status of the system returns to the settings as in point 2 (FIG. 3);
e) the pump is being stopped;
f) PSCM generates the signal ‘PRESSURE READY’.

Preferably, in stage (v):
a) all valves are closed;
b) the pump is turned off;
c) the measurement of optical signal is turned on;
d) PSCM generates the signal ‘MEASUREMENT’.

Preferably, in stage (vi):
a) the pump is turned on;
b) valves 1÷3 are closed;
c) valves 4 and 5 are open;
d) the baratron monitors the pressure in the multipass cell and in the preliminary container;
e) when the pressure reaches the value of 0.001 atm, the valves and the pump are turned off and the system switches to the status as in 1 (FIG. 3);
f) PSCM generates the signal ‘READY’.

The invention also provides a system for the detection of gases, especially biomarker gases, which uses multiplexing and demultiplexing of optical signals, which system comprises at least two lasers in which the light optical paths are directed to an element combining the laser light beams, selected from among an optical splitter, polarization splitter or 50/50 splitter, which is subsequently connected by a coaxial optical path to a multipass cell comprising at least two concave mirrors arranged in parallel, and a gas inlet and outlet, which is filled with a sample of the analyzed gas, the element combining the laser light beams is also connected by an optical path with a cell with reference gases and an inlet photodetector (IN) in front of the multipass cell;

behind the multipass cell there in an outlet photodetector (OUT) connected thereto by an optical path, characterized in that the system comprises only one inlet photodetector (IN) which is connected by an optical path for coaxial directing with the combined laser light beams by the element combining light beams, and only one outlet photodetector (OUT) to which the beams from the multipass cell are directed, wherein each laser is electrically connected to a modulator assigned to that laser, and the modulator is electrically connected to an independent lock-in phase converter, and the lock-in phase converters are electrically connected to the outlet photodetector (OUT) and the inlet photodetector (IN).

Preferably, the said system comprises at least two lasers, and the element combining the laser light beams is at least one optical coupler connected to the laser by means of an optical fiber. The optical fiber has the role of an optical path.

Preferably, the inlets of the multipass cell and the cell containing the reference gases are connected by means of optical paths to the coupler and there is a collimator in each of the paths.

Preferably, the lasers are lasers emitting monochromatic light beams of different wavelengths.

Preferably, the distance between the mirrors in the multipass cell is at least 1 cm, preferably 50 cm.

Preferably, each laser light beam, before the coaxial multiplexing of the beams into one stream, is amplitude modulated by means of a modulator, each with a different frequency.

Preferably, each laser light beam, before the coaxial multiplexing of the beams into one stream, is modulated by means of the modulator with an appropriate wavelength (FM) of different frequencies, each with different frequency.

Preferably, one of the beams leaving the element combining the laser beams, connected by the optical path with the inlet photodetector first travels through the cell filled with the reference gases.

The inventions as defined above enable detection of any biomarker in the air exhaled from patient's lungs in real time with the use of a laser that emits radiation having a wavelength adjusted to the absorption spectrum of the biomarker sought. Preferably, such a biomarker is a gaseous biomarker (biomarker gas) selected in particular from: ethane, methane, formaldehyde and water vapor, as described in the examples below.

Preferably, the system enables simultaneous detection of a number of biomarkers equal to the number of lasers used.

The invention will be described below in a preferable example with reference to the attached drawings wherein:

FIG. 1 shows a general diagram of the structure of the measurement system;

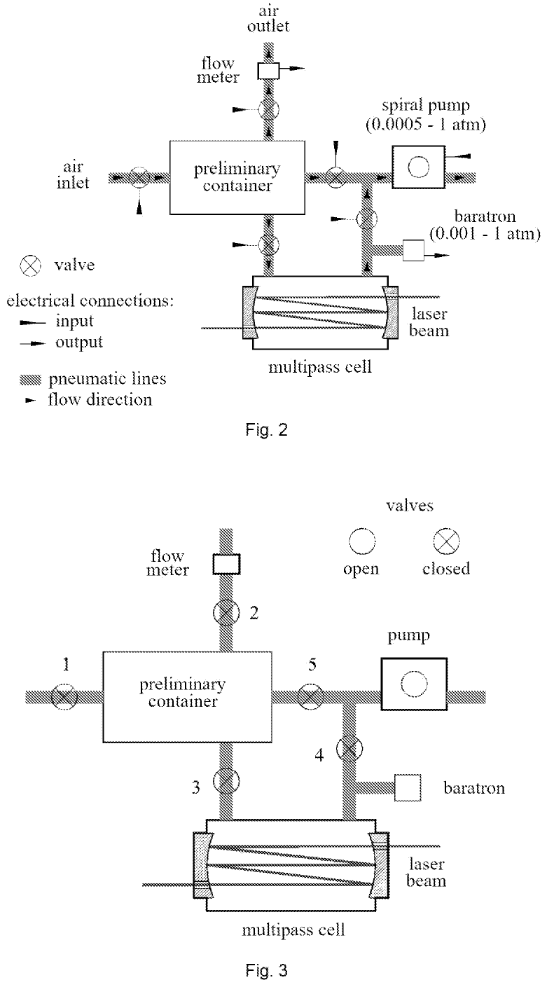

FIG. 2 shows a general diagram of the pneumatic system;

FIG. 3 shows a diagram of the pneumatic system when being prepared to take a sample of breath and measure a biomarker;

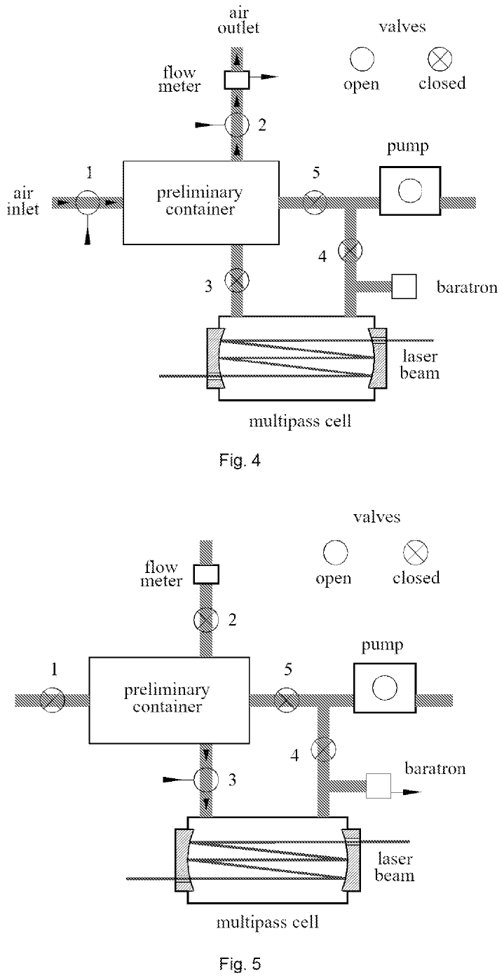

FIG. 4 shows a general diagram of the pneumatic system when taking a sample of patient's breath;

FIG. 5 shows a general diagram of the pneumatic system when filling the multipass cell;

FIG. 6 shows a general diagram of the pneumatic system when adjusting the pressure in the multipass cell;

FIG. 7 shows a general diagram of the pneumatic system when emptying the pneumatic system;

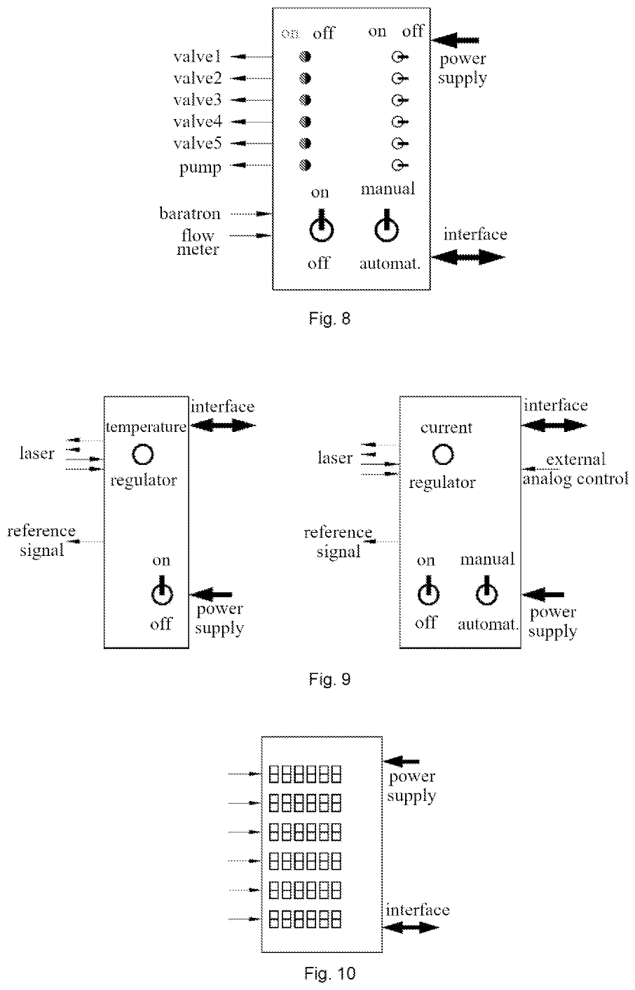

FIG. 8 shows a general diagram of the pneumatic system control module;

FIG. 9 shows a general diagram of the laser operation control module;

FIG. 10 shows a general diagram of the laser status monitoring module;

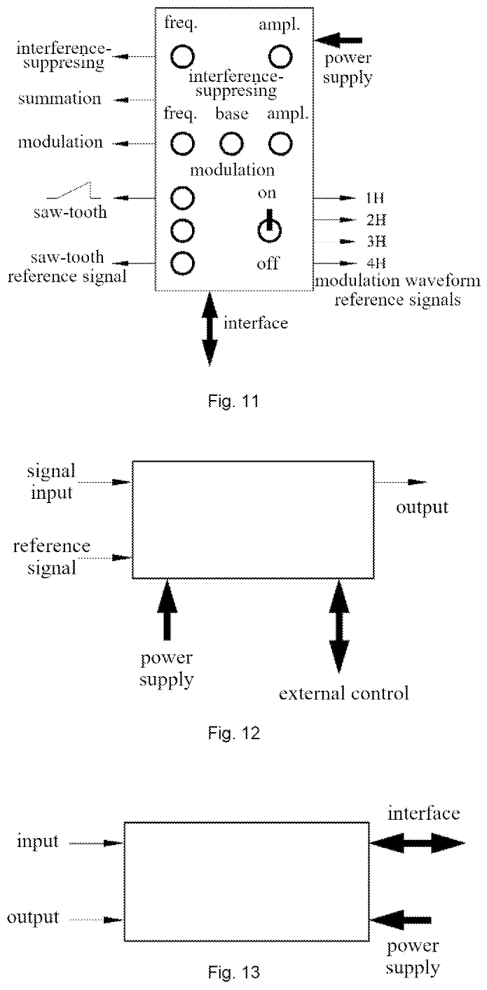

FIG. 11 shows a general diagram of the generator module;

FIG. 12 shows a general diagram of the phase detector module;

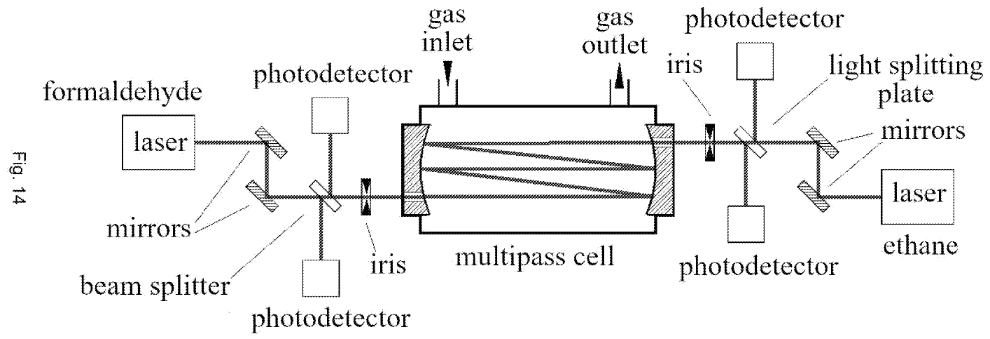

FIG. 13 shows a general diagram of the analogue-digital converter module;

FIG. 14 shows a general diagram of an alternative multipass cell comprising two lasers;

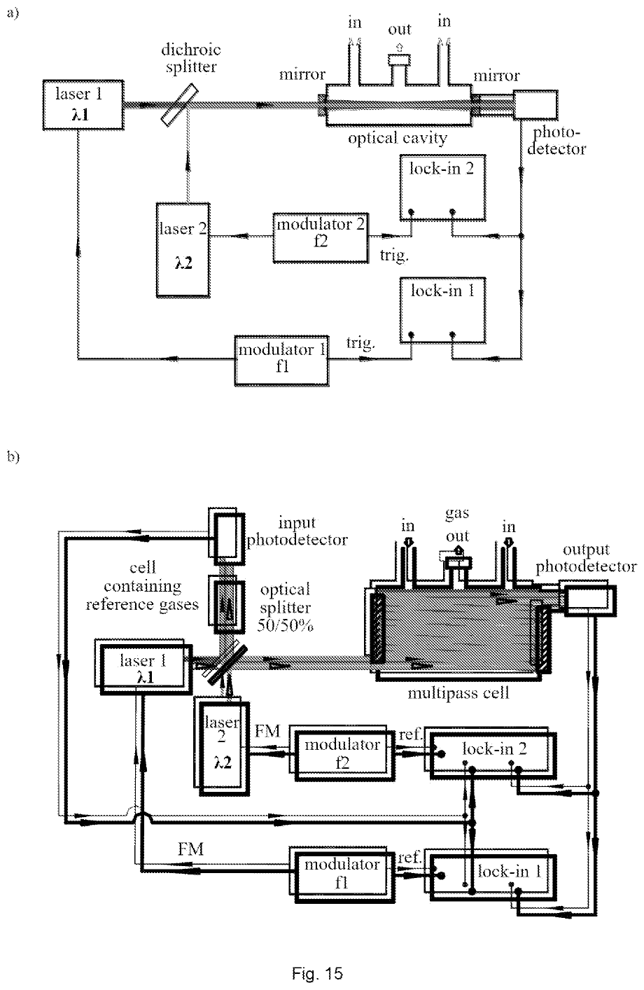

FIG. 15 shows a diagram of a double-beam system for CRDS laser spectroscopy with AM modulation and for multipass spectroscopy with FM modulation;

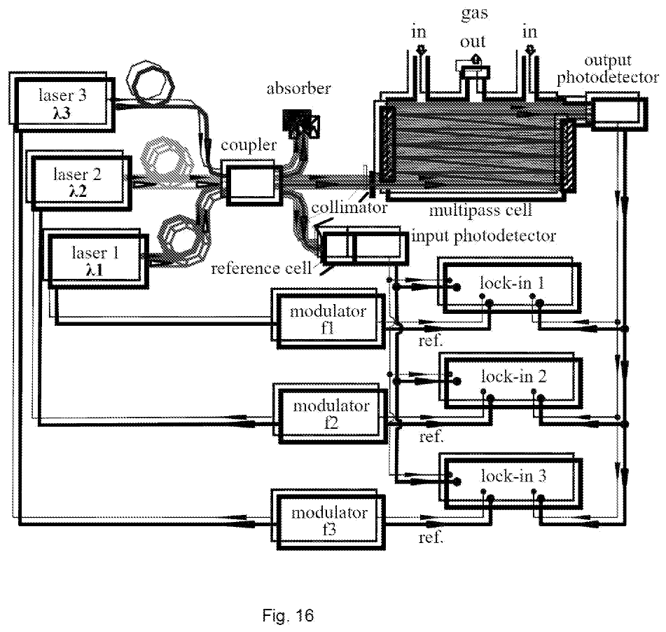

FIG. 16 shows a diagram of a triple-beam system for laser multipass spectroscopy with FM modulation;

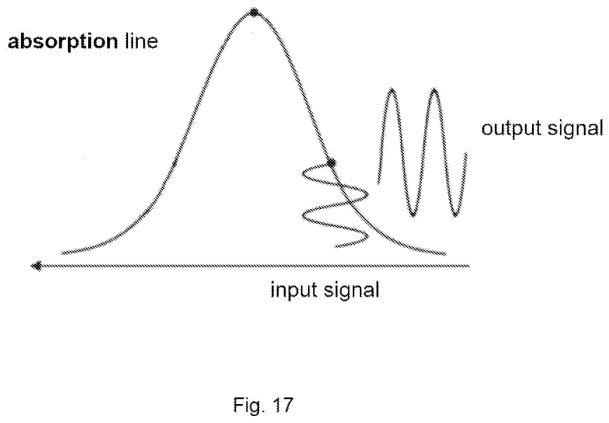

FIG. 17 shows a switch of FM signal to AM signal on the edge of the absorption line.

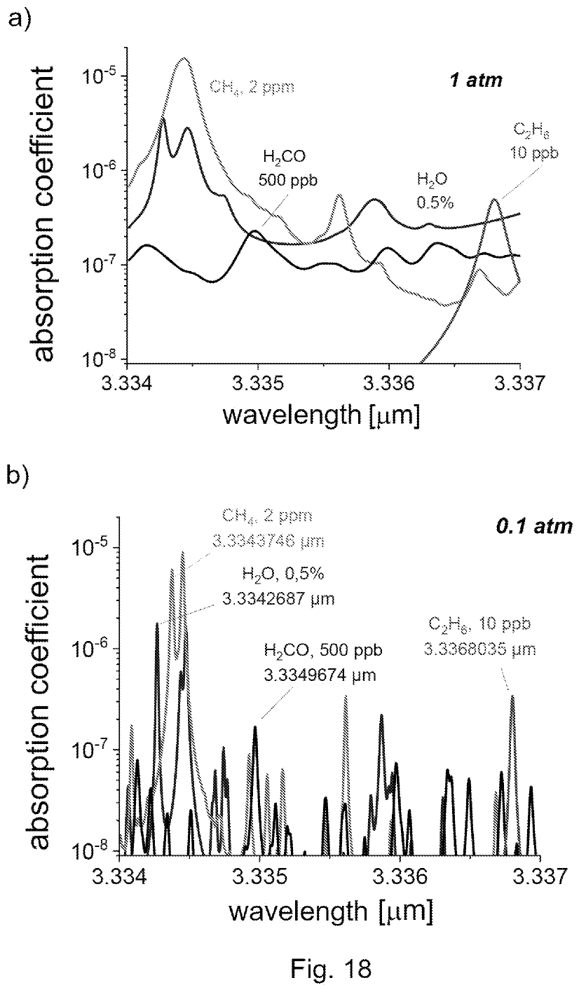

FIG. 18 shows absorption spectra obtained for ethane and main interferents present in the air under the pressure of a) 1 atm; b) 0, 1 atm. In the calculations of spectra based on HITRAN database, the absorption trajectory having the length of 17.5 m have been assumed (Rothman, L. S., Gordon, I. E., Babikov, Y., Barbe, A., Benner, D. C., Bernath, P. F., & Wagner, G. (2013). The HITRAN2012 molecular spectroscopic database. Journal of Quantitative Spectroscopy and Radiative Transfer, 130, 4-50).

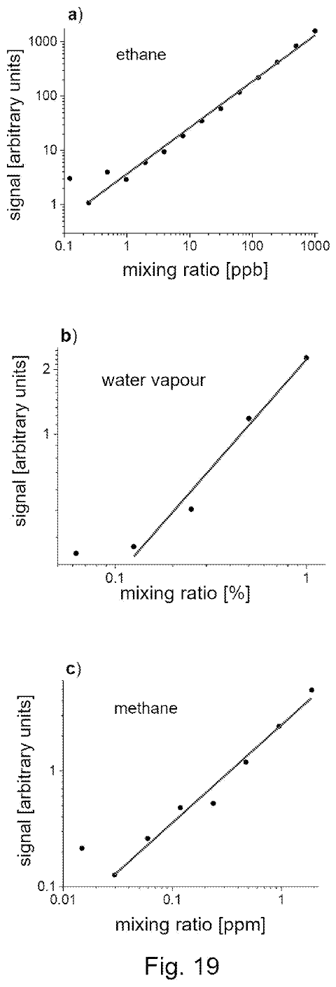

FIG. 19 shows the results of measurements of ethane (a), water vapor (b) and methane (c) obtained in accordance with the invention;

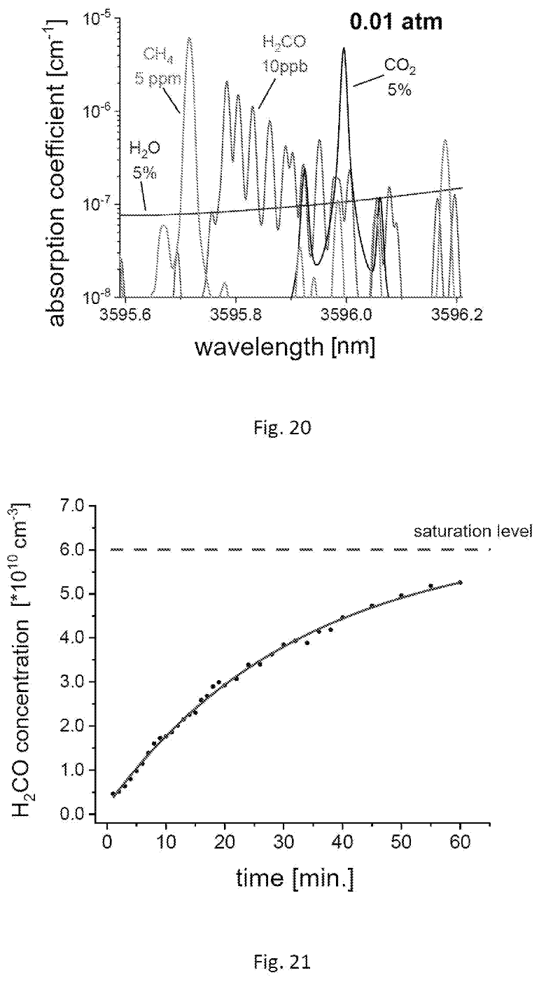

FIG. 20 shows the absorption spectrum of formaldehyde and typical interferents present in breath at pressure reduced to 0.01 atm.

FIG. 21 shows an increase in formaldehyde concentration as a result of polyoxymethylene evaporation from the walls with matched exponential growth (equation 2).

### PREFERABLE EXAMPLES OF INVENTION

The invention consists in the use of coaxial modulated laser radiation beams, wherein a different modulation frequency is used for each wavelength. Having travelled through a spectroscopic system (e.g. an absorption cell) the beam is registered by one common photodetector. The measurement of intensity at each wavelength (demultiplexing) is performed with the use of an independent phase detection system controlled by an appropriate reference signal from a laser modulating generator. The invention enables the detection of two (or more) compounds present in the air sample contained in a detection chamber (e.g. a multipass cell).

The examples described below illustrate exemplary systems according to the invention and the method for the detection of biomarkers in the air exhaled from patient's lungs according to the invention. The examples presented below serve to better understand the invention and are not intended to limit its scope in any way. The examples concerning the structure of the system according to the invention confirm its usability in the process of registering optical signals in the laser beam spectroscopy applications according to the invention.

### Example 1

### General Diagram of the Structure of the Measurement System

The basic element of the system is a multipass cell in which a sample of the exhaled air is examined. The cell is a part of the optical system which is presented in FIG. 1 for the detection of one biomarker (e.g. formaldehyde and/or ethane). The optical system also comprises a laser and two photodetectors measuring the intensity of a light beam before it enters the cell and after it has travelled through the cell. Appropriate electronic modules control the laser temperature and laser current thus adjusting the wavelength thereof to the absorption line of the marker sought. A laser operation monitoring module is used to monitor the operation of the modules. The measurement process requires a complex laser tuning, which is carried out by means of the generator module. Signals from the photodetectors are registered by an A/D converter and phase detector. The analysis of signals and determination of biomarker concentration in a sample are performed in an external computer.

An air sample to be tested is delivered to the multipass cell by the pneumatic system. It comprises a preliminary container, a set of valves with elastic connections and a spiral pump. The system is controlled by means of an appropriate electronic module controlled from the computer by means of an interface.

The control electronics system provides that, in addition to signal connections, information is transmitted between modules with the use of an internal interface (SPI) by means of a central block of communication with PC. This block connects to the computer by a USB connector.

A more detailed description of individual component elements of the measurement system is included below in the following examples of the invention.

### Example 2

### Description of the Proper Operation of the Pneumatic System and General Information Concerning the Pneumatic System.

The pneumatic system comprises:
a) a preliminary container
b) a gas flow meter
c) a multipass cell
d) a pressure meter (baratron)
e) an oil-free spiral pump.

The subassemblies are interconnected by means of elastic gas lines. Between them are located electrically controlled valves, normally closed when at rest. The gas flow meter and baratron provide information (output) to the pneumatic system control module (PSCM) by means of electrical connections. The valves and the pump receive control signals from the system. When they are turned on, the system control circuit generates appropriate logical signals (VALVE 1÷VALVE 2 and PUMP).

### Pneumatic System Operation Phases

### 1. Preparing the System for Breath Sampling and Measuring a Biomarker:

a) the preliminary container is filled with air at atmospheric pressure;
b) there is no gas flow through the flow meter;
c) the multipass cell is pumped out down to the pump pressure limit (0.001 atm);
d) the pump is turned off;
e) all valves (1-5) are closed;
f) PSCM generates the signal ‘READY’.

### 2. Breath Sampling:

a) valves 1 and 2 are open;
b) the remaining valves (3÷5) are closed;
c) the pump is turned off.

### Direct Sampling:

d) the air to be examined is blown into the inlet through a spirometry mouthpiece;
e) excess air escapes through the flow meter and the outlet;
f) the flow meter informs the system control circuit about the gas flow rate;
g) taking into account the flow rate and time, PSCM generates the signal ‘BREATH READY’.

Sampling by means of an external sampling system. Points 2a÷2e are carried out. The signal of an appropriate phase of breath is delivered to the control circuit from an external system. PSCM generates the signal ‘BREATH READY’.

In each case, when the signal ‘BREATH READY’ has been displayed, valves 1 and 2 are closed (FIG. 3). After that process, the preliminary container is filled with the sample of exhaled air at atmospheric pressure.

### 3. Filling the Multipass Cell:

a) valves 1, 2 and 4 and 5 are closed;
b) valve 3 (with a flow limiter) is being opened. Gas from the preliminary container flows to the multipass cell (FIG. 5);
c) pressure build-up in the multipass cell is monitored by the baratron. In the case when 0.2 atm is exceeded, valve 3 is being closed 3, and the status of the system returns to the settings as in point 2 (FIG. 3);
d) PSCM generates the signal ‘FILLING READY’.

### 4. Adjusting Pressure in the Multipass Cell:

a) valves 1÷3 and 5 are closed;
b) valve 4 (with a flow limiter) is being opened;
c) the pump is being turned on—the pressure in the multipass cell is being reduced (FIG. 6);
d) the pressure in the multipass cell is monitored by the baratron, when it reaches the measurement value (e.g. 0.1 atm for ethane and 0.01 atm for formaldehyde) valve 4 is being closed and the status of the system returns to the settings as in point 2. 2 (FIG. 3);
e) the pomp is being stopped;
f) PSCM generates the signal “PRESSURE READY’.

### 5. Measuring the Content of Biomarkers in the Sample:

a) all valves are closed;
b) the pump is turned off;
c) the measurement of optical signal is turned on; PSCM generates the signal “MEASUREMENT”.

### 6. Emptying the Pneumatic System:

a) the pump is turned on;
b) valves 1÷3 are closed;
c) valves 4, 5 are open;
d) the baratron monitors the pressure in the multipass cell and in the preliminary container;
e) when the pressure reaches the value of 0.001 atm, the valves and the pump are being turned off and the system returns to the status as in point 1 (FIG. 3);
f) PSCM generates the signal ‘READY’.

### Example 3

Description of the electronics controlling the pneumatic system

### General Information

The controlling electronics is constructed in the form of modules. The modules are structured in the blocks of the RACK 19 cassette.

### 1. Pneumatic System Control Module (PSCM):

a) provides operation of valves 1÷5 and the pump;
b) turning-on of the valves and the pump is confirmed by the light of a green diode;
the turned-off status is confirmed by the light of a red diode;
c) the valves and the pump can also be activated manually by means of respective switches when the operation mode switch is in the ‘manual” position, or automatically, through the interface—the switch in the ‘automat.’ position.
d) the module receives analog signals from the baratron and flow meter and transmits them in a digital form to the interface;
e) the operation of the pneumatic system control module is controlled by means of an internal microprocessor;
f) the module can be completely turned off (off) by means of the main switch (bottom left corner).

### 2. Laser Operation Control Module:

### Laser Temperature Control Module

a) temperature setpoint-mainly manual;
b) the module produces the reference intensity used for monitoring the temperature;
c) the module generates information about the temperature of the laser for the IT system, transmitted through the interface.

### Laser Current Control Module

a) current setpoint-manual and remote by means of a voltage signal delivered to the ‘analog external control’ input;
b) the module produces analog reference voltage used for monitoring the current;
c) the module generates information about the laser current for the IT system, which is transmitted by the interface;
d) the module enables the control of the laser current by the IT system by means of the interface.

### Each Laser is Controlled by the Above Two Modules.

### 3. Laser Status Monitoring Module:

a) a set of precise voltmeters having 5½ digit accuracy;
b) they are designed to monitor analog reference voltages from the laser current and temperature control modules;
c) each laser requires two voltmeters;
d) additionally, there are provided: (i) the monitoring of radiation intensity and (ii) the monitoring of other voltages important for the system;
e) the module produces information which is transmitted to the system by means of the interface.

### 4. The Generator Module Generates the Following Signals:

a) sinusoidal, the interference-suppressing optical system with adjustable frequency and amplitude;
b) sinusoidal, modulating the wavelength of lasers with adjustable amplitude, frequency and constant background;
c) saw-tooth signal with adjustable amplitude, frequency and constant background;
d) the sum of the modulating, interference-suppressing and saw-tooth signals;
e) four harmonics of the modulating signal in TTL standard (1H÷4H-reference signals);
f) reference saw-tooth signal in TTL standard;
g) the module produces information transmitted to the system by the interface;

h) output resistances—50Ω.

### Detection of Each Biomarker Requires an Independent Generator Module.

### 5. Phase Detector Module:

a) receives the signal from the photodetector measuring the intensity of radiation travelling through the multipass cell, and (as the reference signal) TTL signal corresponding to the selected harmonic of the laser modulation signal;
b) produces voltage proportional to the amplitude of given harmonic in the signal from the photodetector.

### Detection of Each Biomarker Requires an Independent Phase Detection Module.

### 6. Analog-Digital Processor Module:

a) receives the signal from the photodetector monitoring laser intensity before entrance to the multipass cell;
b) receives the output voltage from the lock-in detector;
c) sends the signal in the form of the absorption spectrum of the given biomarker through the interphase to the computer.

### Detection of Each Biomarker Requires an Independent Analogue-Digital Converter Module.

### 7. External Computer (PC) Communication Module:

a) communicates with the PC by means of an USB connector;
b) carries out the transmission/receipt of information to/from individual blocks by means of an internal link (SPI).

### Example 4

### Alternative Structure of the Optical System

FIG. 14 shows an alternative (target) optical system for the detection of two (or more) biomarkers in the air exhaled from the lungs. Compared to the system presented in FIG. 1, it uses two lasers and, for each laser, two photodetectors and two systems introducing the light beam to the multipass cell.

### Example 5

The structure of the optical system is based on multiplexing and demultiplexing of the optical signal.

The invention consists in the analysis of the patient's exhaled air by means of an optical system based on combining the laser beams of beam splitters or, when a fiber optic technology is used, optical couplers. Modulation of laser beams at a different frequency for each laser enables demultiplexing by the use of phase detection, independent for each channel, and only one photodetector is used to register light intensity. Schematic diagrams of these solutions for the systems in which CRDS and multipass method are used are presented in FIG. 15. To register radiation intensity for each wavelength an independent phase detector, using a reference signal appropriate for the modulation of the given laser, is used.

Modulation of laser light may be twofold: it may apply to amplitudes (AM) or laser wavelength (FM). In the first case, light intensity changes in time t in the following way:

$${l}_{λ i} ( t ) = {l}_{0 λ i} [ 1 - cos ( 2 π {f}_{i} t ) ] ,$$

in the second case—the laser wavelength changes:

$${λ}_{i} ( t ) = {λ}_{0 i} + {Δλ}_{i} cos ( 2 π {f}_{i} t ) .$$

An example of use of this solution for AM modulation is presented in FIG. 15a. The radiation intensity of each laser (operating at wavelength λ1 and λ2, respectively) is obtained with an independent frequency (f1 and f2, respectively). Having travelled through the optical system (in this case a CRDS resonator, but it may also be a multipass cell with an absorber, or another system changing the intensity of individual beams), the light is registered by the photodetector and directed to phase voltmeters, the number of which equals the number of detection channels. Since the phase voltmeters register only signals with modulation frequency that is accordant with the frequency of the respective modulator, information about the intensity of beams having individual wavelengths is split, i.e. demultiplexed.

Modulation of laser wavelength (FM) is carried out (for example) in spectroscopic systems designed to determine absorption. If FM modulation takes place within the range of wavelengths corresponding to the edge of the absorption (FIG. 17), the signal, having travelled through the absorber, is amplitude modulated. In the case of the example presented in FIG. 15b, FM modulation was used with respect to the multipass system; however, this technique is used for many other applications (also including CRDS). In the system presented, multiplexing enables simultaneous determination of the absorption coefficient of two absorbers. Since it is necessary to measure the intensity of light entering the spectroscopic system at each wavelength, an input photodetector has been used (photodetector in), in front of which there is located a reference cell (ref. cell) with the absorbers sought. Each laser is tuned to the edge of the line of individual absorber, which enables converting FM modulation to AM modulation and measuring the intensity of individual beams by means of appropriate phase voltmeters. Similar demultiplexing of signals takes place after passage through the multipass cell. In this case it is necessary to use two-input phase voltmeters. In the case when a fiber optic technique is used, it is possible to combine a great number of beams (which is difficult to carry out in the case of glass dichroic splitters-prior art). One example of such a system for multipass spectroscopy is presented in FIG. 16.

Combination of the light of three lasers having three wavelengths was possible due to the use of a three-input optical fiber coupler. Usually, it is also possible to use an appropriate number of two-input couplers (in this case two couplers) for this purpose. One of the coupler outputs may be used for determining radiation intensities at the input to the spectral system in the system: fiber optic collimator—ref. cell—photodetector in. Radiation from the second output is directed—through the collimator—to the multipass cell. Having travelled through the cell, radiation is demultiplexed in the above described way. The third output of the coupler (if it exists) is not used; its output is directed to a radiation absorber.

### Example 6

Calibration of the optical system for the detection of biomarkers according to the invention—using as the example of a system for detection with the one with following sensors: ethane, methane, water vapor and formaldehyde.

In order to optically identify biomarkers, i.e. trace gases or gas biomarkers, with high sensitivity, it is necessary to select a strong absorption line in the spectrum of the examined compound. It is also necessary to avoid interference of this line by other compounds that may be present in the examined sample. It is particularly important when analyzing a breath in which 3000 different components have already been detected in the exhaled air. Ethane spectrum consisting of 9 strong lines is within the range of 3.3325-3.3630 μm. The inventors have selected for the detection of ethane the line 3.3368 μm. Although it is not characterized by the strongest maximum in the ethane spectrum, the inventors have found that it has the least interferences.

The main compounds whose spectra may overlap the line of ethane (C₂H₆) are water vapor, methane (CH₄) and formaldehyde (H₂CO). At atmospheric pressure, the shapes of the lines are mainly determined by the effect of collision line broadening occurring at a high atmospheric pressure. (FIG. 18a). Wide spectra of H₂O, CH₄ and H₂CO prevent a high sensitivity of ethane measurement in such conditions (FIG. 18a). Lowering the air pressure in a sample to 0.1 atm effectively reduces this broadening and leads to the separation of lines (FIG. 18b), and there is much less interference with the C₂H₆ absorption peak (3.3368035 μm). The analysis of this spectrum leads to the conclusion that it is possible in such a situation to detect ethane with the sensitivity of about 1 ppb. Therefore, it is necessary to use a reduced pressure of the sample in the measurement system and consequently use appropriate vacuum pumps, valves and pressure sensors (baratron).

In order to examine the sensitivity and linearity of the system according to the invention, gradual dissolution of calibrated reference mixture has been used (Messer, 1 ppm C₂H₆ in nitrogen). In the first stage, the mixture was introduced to the multipass cell at 0.1 atm. When the signal was collected, the mixture was diluted with pure nitrogen (evaporated from its liquid phase). The pressure was elevated to 0.2 atm. After the period of about 15 min., necessary to obtain good mixing of the gases, the pressure was again reduced to 0.1. atm. This produced ethane mixture having a twice lower density than the previous value. The signal was again registered. Subsequent actions of diluting and signal registering were repeated. The optical determination of C₂H₆ concentration was based on the comparison of amplitudes for a specific concentration with an amplitude of the signal delivered by reference mixture.

Data collected in this way are presented in FIG. 19a. The sensor was operating in a linear manner up to the concentration of 1 ppb (about 2.7·10⁹ cm⁻³). An Allan deviation graph was also made for this concentration (Cutler, L. S., & Searle, C. L. (1966). Some aspects of the theory and measurement of frequency fluctuations in frequency standards. Proceedings of the IEEE, 54(2), 136-154.). The precision of about 70 ppt was achieved for the optimum integration time of 20 s. This means that the detection threshold of 1 ppb achieved in practice is sufficient for the examination of the air exhaled from patient's lungs, because for ethane the disease concentration threshold is about 3.5 ppb.

The system according to the invention constructed in this way is designed primarily for a sensitive detection of ethane; nevertheless—as can be seen in FIG. 18b—also weak lines of other compounds are present within the tuning scope of the laser used. Thus, the system according to the invention can also be used for the detection of those compounds (biomarkers), but with correspondingly lower sensitivity.

To calibrate the system with a water vapor sensor discussed herein, water present in the ambient air was used. It was assumed to be the reference concentration. Knowing the air temperature and relative humidity one is able to define the concentration of H₂O by means of Magnus formula (Alduchov, O. A., & Eskridge, R. E. (1996). Improved Magnus form approximation of saturation vapor pressure. Journal of Applied Meteorology and Climatology, 35(4), 601-609). The temperature of 23° C. and relative humidity of 35% were registered during the experiment in the laboratory. This corresponded to 2.4·10¹⁷ cm⁻³ of H₂O molecules. Detection was performed on the line 3.3342687 μm (FIG. 18b). In this case the procedure of serial dilutions was also used. The results of measurements are presented in FIG. 19b. The system operated linearly up to H₂O dilution level of about 0.1 (2.4·10¹⁶ cm⁻³ in normal conditions).

A relatively high absorption coefficient of methane (FIG. 18b) also enables detection of this component. The inventors tested the herein discussed system with the methane sensor in respect of methane detection in the line 3.3343746 μm. Since a calibrated mixture was not available, the mean CH₄ concentration in the ambient air (1.9 ppm) was assumed to be the approximate reference concentration. The results are presented in FIG. 19c. The sensor operated linearly up to the dilution level of 1/64. This means that the useful level of methane detection in the system according to the invention is about 30 ppb.

In this study the inventors have demonstrated the novel system for direct detection and measurement of a biomarker in the air exhaled from human lungs, using ethane as an example. The obtained sensitivity is sufficient for clinical tests. Additionally, the system provides the possibility of measuring other biomarkers, such as methane, which allows one to study correlations between those marker compounds in breath.

In the case of formaldehyde, the measurement was performed at 0.01 atm pressure and with a laser emitting radiation within the wavelength range of 3595.77-3596.20 nm, which results from the properties of the absorption spectrum of that compound. The results are presented in FIG. 20.

In this case, the method for measuring the sensitivity and linearity of the operation of the system according to the invention by filling it with mixtures of air with the examined biomarker in decreasing concentrations, in this case formaldehyde, does not work out because, unfortunately, one should expect the effect of formaldehyde adhering to the walls of the apparatus in another form of this compound, polyoxymethylene, which has a solid physical state. It is should be noted that ethane does not have such a property; it does not adhere to the walls. Nevertheless, polyoxymethylene may also evaporate from the walls and fill the sensor (FIG. 21). It is disadvantageous because evaporated, gaseous, formaldehyde interferes with the measurement of that compound in a sample to be examined. However, this can be overcome using a dynamic method, and that is what the inventors have done in order to determine the sensitivity of the system according to the invention. A simple model of H₂CO deposition on the sensor walls and its evaporation has been developed. In this approach, the density of evaporating molecules in a unit of time is equal to a, whereas the reverse process, that is the number of molecules adhering to the surface of the apparatus is proportional to the concentration of H₂CO (n) vapor and deposition constant k. The evolution of formaldehyde vapor concentration is subsequently described by the differential equation:

$$\frac{dn ( t )}{dt} = - k n ( t ) + α . \qquad ( 1 )$$

Its result for the initially empty sensor has the following form:

$$n ( t ) = \frac{α}{k} [ 1 - exp ( - kt ) ] . \qquad ( 2 )$$

This function fits well with the experimental data. (FIG. 21). The fitting parameters were: deposition constant k=5·10⁻⁴ s⁻¹, evaporation rate α=3·10⁷ cm⁻³ s⁻¹ and saturation concentration: a/k=6·10¹⁰ cm⁻³.

Using the above equation 2 and fitting parameters, one can assess that due to evaporation of polyoxymethylene, the sensor was filled from immeasurable H₂CO concentration (at the initial moment) to the concentration of about 1.8·10⁹ cm⁻³ within the first minute of the measurement. This corresponds to the mixing ratio of about 6.6 ppb.

Thus, the measurement of formaldehyde concentration in the air is a ‘dynamic’ measurement; it consists in pumping out the sensor by pumping out to the pressure of about 10⁻⁴ atm and introducing therein the examined air sample under 0.01 atm pressure. Since it was assumed that, due to the averaging of signals, the time of formaldehyde measurement by means of the studied system, necessary to eliminate the interference, is about 1 min, the value of 6.6 ppb has been assumed to be the threshold concentration of formaldehyde detection for the system according to the invention. In practice, this time is often shorter, even 3 times shorter; thus the sensitivity of the system is thus 3 times higher.

1. A system for the detection of biomarkers in the air exhaled from patient's lungs, comprising a leak-proof multipass cell provided with a gas inlet and outlet into which a sample of the exhaled air is introduced by means of a pneumatic system, wherein the multipass cell is provided with one laser whose light beam is directed along a coaxial optical path to the multipass cell, comprising at least two concave mirrors arranged in parallel, and a gas inlet and outlet, which is filled with a sample of the analyzed exhaled air, wherein the pneumatic system is connected to the gas inlet and outlet of the multipass cell by means of lines supplying the exhaled air, characterized in that the pneumatic system comprises a preliminary container of the exhaled air comprising an air inlet and air outlet, wherein the air outlet is additionally provided with an air flow meter, the preliminary container is further connected by an elastic line supplying the air with the multipass cell on one side and on the other side with a pump, baratron and air outlet of the multipass cell.

2. The system according to claim 1, characterized in that the pump is a spiral pump, preferably an oil-free pump, adjusted to reduce pressure within the range of 1 atm to 0.0005 atm.

3. The system according to claim 1, characterized in that the baratron is adjusted to measure reduced pressure within the range of 1 atm to 0.001 atm.

4. The system according to claim 1, characterized in that the preliminary container comprises elastic lines supplying the analyzed exhaled air, wherein each line is independently provided with an independently controlled valve.

5. The system according to claim 1, characterized in that the elastic line supplying the analyzed exhaled air additionally comprises a valve between the pump and the baratron.

6. The system according to claim 1, characterized in that the laser is connected by a coaxial optical path to the multipass cell and to one photodetector in front of the multipass cell and another photodetector behind the multipass cell.

7. The system according to claim 1, characterized in that the pneumatic module is controlled by means of a pneumatic system control module of valves and the pump.

8. The system according to claim 1, characterized in that the laser is connected by means of electric lines to a laser temperature control module and a laser operation control module, and both modules are electrically connected to a laser status monitoring module.

9. The system according to claim 1, characterized in that the laser is a laser emitting monochromatic light beams of different wavelengths.

10. The system according to claim 1, characterized in that the laser emitting monochromatic beams is a laser emitting a monochromatic light beam having a wavelength adjusted to the edge of the absorption line of formaldehyde (HCOH) within the range of 3595.77-3596.20 nm and a laser emitting a light beam adjusted to the edge of the absorption line of ethane (C₂H₆) within the range close to 3336.8 nm.

11. The system according to claim 1, characterized in that 1046 Hz rectangular signals are used to modulate the laser emitting light having a wavelength within the range of 3595.77-3596.20, whereas 1429 Hz rectangular signals are used to modulate the laser emitting light having a wavelength within the range close to 3336.8 nm.

12. The system according to claim 1, characterized in that the distance between the mirrors in the multipass cell is at least 1 cm, preferably 50 cm.

13. A method for the detection of biomarkers in the air exhaled from patient's lungs with the use of the system according to claim 1, characterized in that it comprises the following stages:
(i) preparing the system to take a sample of exhaled air and measure a biomarker;
(ii) taking a biomarker sample;
(iii) filling the multipass cell;
(iv) adjusting pressure in the multipass cell;
(v) measuring the content of biomarkers in the air sample; and
(vi) emptying the pneumatic system of the examined air.

14. The method according to claim 13, characterized in that, in stage (i):
a) the preliminary container is filled with the air at atmospheric pressure;
b) there is no flow of gas through the flow meter;
c) the multipass cell is pumped out down to the pressure limit of the pump (0.001 atm);
d) the pump is turned off;
e) all valves (1-5) are closed;
f) PSCM generates the signal ‘READY’, and/or
in stage ii):
a) valves 1 and 2 are open;
b) the remaining valves (3÷5) are closed;
c) the pump is turned off, and/or
in stage (iii):
a) valves 1, 2 are 4 and 5 are closed;
b) valve 3 (with a flow limiter) is being opened and the gas from the preliminary contained flows to the multipass cell;
c) pressure build-up in the multipass cell is monitored by the baratron, and/or
in stage (iv):
a) valves 1÷3 and 5 are closed;
b) valve 4 (with a flow limiter) is being opened;
c) the pump is being turned on—the pressure in the multipass cell is being reduced;
d) the pressure in the multipass cell is monitored by the baratron, and in the case when it reaches the measurement value, valve 4 is being closed and the status of the system returns to the settings as in point 2;
e) the pump is being stopped;
f) PSCM generates the signal ‘PRESSURE READY’, and/or
in stage (v):
a) all valves are closed;
b) the pump is turned off;
c) the measurement of the optical system is turned on;
d) PSCM generates the signal ‘MEASUREMENT’, and/or
in stage (vi):
a) the pump is turned on;
b) valves 1÷3 are closed;
c) valves 4 and 5 are open;
d) the baratron monitors the pressure in the multipass cell and in the preliminary container;
e) when the pressure reaches the value of 0.001 atm, the valves and the pump are being turned off and the system returns to the status as in point 1;
f) PSCM generates the signal ‘READY’.

15.-19. (canceled)

20. A system for the detection of gases, especially biomarker gases, which system uses multiplexing and demultiplexing of optical signals, comprising at least two lasers, in which the light optical paths are connected to an element combining the laser light beams, selected from among an optical splitter, polarization splitter or 50/50 splitter, which is subsequently connected by a coaxial optical path to a multipass cell comprising at least two concave mirrors arranged in parallel and a gas inlet and outlet, which is filled with a sample of the analyzed gas, the element combining the laser light beams is also connected by an optical path to a cell with reference gases and an input photodetector (IN) in front of the multipass cell; behind the multipass cell there is an output photodetector (OUT) connected to the cell by an optical path, characterized in that the system comprises only one inlet photodetector (IN) which is connected by an optical path for coaxial directing with combined laser light beams by means of the element combining the light beams, and only one outlet photodetector (OUT) to which beams from the multipass cell are directed, wherein each laser in electrically connected to a modulator assigned to that laser, and the modulator is electrically connected to an independent lock-in phase converter, and the lock-in phase converters are electrically connected to the outlet photodetector (OUT) and the inlet photodetector (IN).

21. The system according to claim 20, characterized in that it comprises at least two lasers, and the element combining the laser light beams is at least one optical coupler connected to the laser by means of an optical fiber.

22. The system according to claim 21, characterized in that the inputs of the multipass cell and the cell containing reference gases are connected by means of optical paths to the coupler, and there is a collimator in each of those paths.

23. (canceled)

24. The system according to claim 1, characterized in that the distance between the mirrors in the multipass cell is at least 1 cm, preferably 50 cm.

25. The system according to claim 1, characterized in that each laser light beam, prior to the coaxial multiplexing of the beams into one stream, is amplitude modulated by means of a modulator, each with a different frequency, and/or
characterized in that, each laser light beam, prior to the coaxial multiplexing of the beams into one stream, is modulated by means of the modulator with an appropriate wavelength (FM) of different frequencies, each with a different frequency, and/or
characterized in that one of the beams leaving the element combining the laser beams and connected by an optical path to the inlet photodetector, first travels through the cell filled with the reference gases, and/or
characterized in that the lasers are lasers emitting monochromatic light beams of different wavelengths.

26.-27. (canceled)

28. The system according to claim 1, or the method for the detection of biomarkers in the air exhaled from patient's lungs that uses the system, characterized in that the biomarker is selected from among: ethane, methane, formaldehyde and water vapor.
